(19) United States
(12) Patent Application Publication
(10) Pub. No.: US 2023/0223113 A1
(43) Pub. Date: Jul. 13, 2023

(54) Methods and Systems for Rapid Antimicrobial Susceptibility Tests

(71) Applicant: The Regents of the University of California, Oakland, CA (US)

(72) Inventors: Regina Ragan, Irvine, CA (US); Allon Hochbaum, Irvine, CA (US); William John Thrift, San Francisco, CA (US)

(73) Assignee: The Regents of the University of California, Oakland, CA (US)

(21) Appl. No.: 17/999,674

(22) PCT Filed: May 27, 2021
(86) PCT No.: PCT/US21/34652

§ 371 Date: Nov. 22, 2022

Related U.S. Application Data
(60) Provisional application No. 63/030,821, filed on May 27, 2020.

(51) Int. Cl. G16C 20/70

(52) U.S. Cl. CPC G16C 20/70

(57) ABSTRACT

Rapid antimicrobial susceptibility testing (AST) can be an integral tool to mitigate the unnecessary use of powerful and broad spectrum antibiotics that leads to the proliferation of multi-drug resistant bacteria. Methods and systems for a sensor platform composed of surface enhanced Raman scattering (SERS) sensors with surfaces having molecular control of nano architecture and surface chemistry and machine learning processes for analyzing SERS data, are described to detect metabolic profiles from susceptible antibiotic resistant bacteria strains for rapid AST.

### CROSS-REFERENCE TO RELATED APPLICATIONS

The current application claims the benefit of priority to U.S. Provisional Patent Application No. 63/030,821 entitled “Methods and Systems for Rapid Antimicrobial Susceptibility Tests Using Optical Nose” filed May 27, 2020. The disclosure of U.S. Provisional Patent Application No. 63/030,821 is hereby incorporated by reference in its entirety for all purposes.

### STATEMENT OF FEDERALLY SPONSORED RESEARCH

This invention was made with Government support under Grant No. EECS1449397, awarded by the National Science Foundation. The Government has certain rights in the invention.

### FIELD OF THE INVENTION

The present invention generally relates to methods and systems for rapid antimicrobial susceptibility tests; and more particularly to methods and systems that utilize olfactory-like sensing platform integrating surface enhanced Raman scattering sensors and machine learning computing to determine effective antibiotic therapy.

### BACKGROUND

Antimicrobial resistance (AMR) may cause deaths of hundreds of thousands people annually with bacterial infections. AMR can be exacerbated by the unnecessary prescription of broad spectrum antibiotics. A full third of antibiotics prescribed are to treat bacteria that are resistant to those therapeutics, or which may be otherwise inappropriate. While AMR can be a multifaceted problem that may require many systemic changes to healthcare, rapid diagnostics to reduce the unnecessary use of antimicrobials as an intervention for the reduction of AMR that uses rapid antimicrobial susceptibility testing (AST) might offer an effective solution.

### BRIEF SUMMARY

Methods and systems in accordance with various embodiments of the invention enable the determination of antibiotic therapy based on rapid antimicrobial susceptibility tests. In many embodiments, antimicrobial susceptibility tests can detect and quantify antibiotic resistant and susceptible bacteria metabolites profile near or below ng/mL concentrations in complex media. Several embodiments provide that antimicrobial susceptibility tests (AST) can differentiate between antibiotic susceptibility and/or resistance of bacteria in clinical infections in less than one hour.

Many embodiments detect differences in bacterial metabolites as a sensing platform for diverse analytes of interest. Bacterial cells can be used as a signal amplification platform for sensing applications combined with machine learning data analysis processes in accordance with several embodiments. Antibiotics that are effective against a given strain of bacteria can induce large changes in metabolite profiles of the cells. Some embodiments detect these changes in the metabolite profiles with sensitivity and accuracy to determine antibiotic effectiveness. In a number of embodiments, analytes that may affect bacterial metabolism including (but not limited to) toxic metals and pesticides, can be detected by analyzing bacterial metabolite changes.

One embodiment of the invention includes a method of rapid antimicrobial susceptibility testing comprising obtaining a set of metabolic profile for at least one bacteria strain using a sensing platform; generating a set of surface enhanced Raman scattering (SERS) spectra based upon the set of bacterial metabolic profile using at least one SERS sensor from the sensing platform; evaluating at least one spectrum based on the set of SERS spectra using a machine learning model implemented on the sensing platform; and when the at least one evaluated spectrum satisfies at least one criterion by the sensing platform, determining at least one antimicrobial susceptibility property of the at least one bacteria strain.

In a further embodiment, the at least one bacteria strain is selected from the group consisting of Pseudomonas aeruginosa (P. aeruginosa), Escherichia coli (E. coli), uropathogenic strain of E. coli, Enterococcus faecalis (E. faecalis), Klebsiella pneumoniae (K. pneumoniae), co-culture of E. coli, K. pneumoniae, and P. aeruginosa, co-culture of E. coli and Salmonella enterica serovar Typhimurium (S. typhimurium), pairwise co-culture of uropathogenic strain of E. coli, E. faecalis, and K. pneumoniae.

In another embodiment, the at least one antimicrobial susceptibility property is selected from the group consisting of antibiotic susceptible metabolite profile, antibiotic resistance metabolite profile, antibiotic temporal response, and antibiotic dosage response.

In still another embodiment, the determination of antibiotic dosage response is at least 10 times lower than a minimum inhibitory concentration.

In a yet further embodiment, the machine learning model is selected from the group consisting of variational autoencoder (VAE), support vector machine (SVM), convolutional neural networks (CNNs), and Bayesian Gaussian mixture.

A still further embodiment includes processing the set of SERS spectra by smoothing, background subtraction, and scaling.

In a further embodiment again, determining when the determined at least one antimicrobial susceptibility property satisfies at least one criterion further comprises, generating a set of SERS spectra based upon the set of metabolic profile for each of the candidate bacteria strain; determining at least one antimicrobial susceptibility property for each of the candidate bacteria strain based on the set of SERS spectra of each of the candidate bacteria strain using the machine learning model; screening the candidate bacteria strain based upon the at least one antimicrobial susceptibility property determined for each of the candidate bacteria strain; and identifying the antimicrobial susceptibility property based upon the screening.

In another additional embodiment, training the machine learning model to learn relationships between the set of SERS spectra and antimicrobial susceptibility properties using a training dataset describing a plurality of bacteria strains and their antimicrobial susceptibility properties.

In another embodiment again, training the machine learning model to learn relationships between the set of SERS spectra and antimicrobial susceptibility properties further comprises obtaining a set of SERS spectra for each bacteria strain in the training dataset of bacteria strains by determining a set of metabolic profile.

In a further additional embodiment, training of the machine learning model is unsupervised, semi-supervised, supervised, or combinations thereof.

In a still yet further embodiment, the machine learning model is a variational autoencoder model and the set of SERS spectra is encoded in the VAE model in to a latent space as Gaussian distributions with mean and variance during training.

Still another additional embodiment includes a method of training a machine learning model to predict at least one antimicrobial susceptibility property from a set of metabolic profile for a bacteria strain comprising: obtaining a training dataset of bacteria strains and their antimicrobial susceptibility properties using a computer system; generating a set of surface enhanced Raman scattering (SERS) spectra for each bacteai strain in the training dataset based upon a set of metabolic profile for each of the candidate bacteria strains using the computer system; training a ML model to learn relationships between the set of SERS spectra of each bacteria strain in the training dataset and the antimicrobial susceptibility properties of each of the bacteria strains in the training dataset using the computer system; and utilizing the machine learning model to predict at least one antimicrobial susceptibility property for a specific bacteria strain based upon a set of SERS spectra generated for the specific bacteria strain based upon a set of metabolic profile for the specific bacteria strain.

In yet another further embodiment, training the machine learning model to learn relationships between the sets of SERS spectra of each bacteria strain in the training dataset and the antimicrobial susceptibility properties of each of the bacteria strains in the training dataset further comprises utilizing a transfer learning process to train a machine learning model previously trained to determine the relationship between a SERS spectrum of a bacteria strain and a different set of antimicrobial susceptibility properties.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIG. 1 illustrates a rapid antimicrobial susceptibility testing (AST) process in accordance with certain embodiments.

FIG. 2 illustrates a convolutional neural network analysis of Rhodamine 800 concentration fro SERS spectra in accordance with prior art.

FIG. 3 illustrates a detection scheme for surface enhanced Raman scattering (SERS) data acquisition and machine learning analysis of bacterial metabolomic samples in accordance with certain embodiments.

FIG. 4 illustrates a scheme for using a variational autoencoder (VAE) for surface enhanced Raman scattering spectra analysis in accordance with certain embodiments.

FIGS. 5A-5C illustrate standard growth curves used to identify the minimum inhibitory concentration of carbenicillin, rifampicin, and gentamicin for Pseudomonas aeruginosa (P. aeruginosa) in contrast to a machine learning analysis in accordance with certain embodiments.

FIG. 5D illustrates a standard growth curve used to identify the minimum inhibitory concentration of gentamicin for Escherichia coli (E. coli) in contrast to a machine learning analysis in accordance with certain embodiments.

FIG. 6A illustrates the optical density at 600 nm (OD600) of a P. aeruginosa cell culture after adjustment to OD 0.5 at time 0 in accordance with certain embodiments.

FIG. 6B illustrates the OD600 of a P. aeruginosa cell culture after adjustment to OD 0.5 and with 50 μg/mL carbenicillin at time 0 in accordance with certain embodiments.

FIG. 6C illustrates the OD600 of a P. aeruginosa cell culture after adjustment to OD 0.5 and with 400 μg/mL rifampicin at time 0 in accordance with certain embodiments.

FIG. 7A illustrates VAE space of AST spectra from untreated, carbenicillin treated, and rifampicin treated P. aeruginosa lysate in accordance with embodiments.

FIGS. 7B-7G illustrate averaged SERS spectra and VAE generated spectra from the center of the class centroid from each untreated, carbenicillin treated, and rifampicin treated P. aeruginosa lysate at different time points in accordance with certain embodiments.

FIG. 8 illustrates a t-stochastic neighbor embedding (t-SNE) visualization of clustering of the SERS spectra in accordance with certain embodiments.

FIGS. 9A-9B illustrate a VAE latent space analysis of SERS spectra of gentamicin dosed P. aeruginosa and E. coli lysate does response at various concentrations of antibiotic, gentamicin, in accordance with certain embodiments.

FIGS. 9C-9D illustrate a VAE latent space analysis of SERS spectra of gentamicin dosed P. aeruginosa and E. coli lysate temporal response at various time points in accordance with certain embodiments.

FIGS. 10A-10E illustrate a semi-supervised SVM model visualization versus the number of training examples from the AST dataset in accordance with certain embodiments.

FIG. 11 illustrates a cycle of data informed transfer learning in accordance with certain embodiments.

FIG. 12A illustrates a VAE latent space from the test portion of the combined metabolite mixture of 2-methyl napthalene (A), o-cresol (B), 2-amino acetophenone (C), pyrrole (D), 2-pentyl furan (E), and indole (F) and AST datasets in accordance with certain embodiments.

FIG. 12B illustrates a t-SNE visualization of a 32-dimensional metabolite combination VAE space in accordance with certain embodiments.

FIG. 13 illustrates isolation forest predictions to discard outliers in AST spectra in accordance with certain embodiments.

FIG. 14A illustrates Bayesian Gaussian Mixture analysis of combined VAE encoded P. aeruginosa AST test spectra in accordance with certain embodiments.

FIG. 14B illustrates a comparison of transfer learning model performance in accordance with certain embodiments.

### DETAILED DESCRIPTION OF THE INVENTION

Turning now to the drawings, methods and systems for rapid antimicrobial susceptibility testing (AST) are described. Many embodiments implement phenotypic AST by detecting and analyzing bacterial metabolic profile. Phenotypic AST can be carried out using a sensing platform. Some embodiments include that the sensing platform can integrate surface enhanced Raman scattering (SERS) sensors with surfaces having molecular control of nano-architecture and surface chemistry. Several embodiments implement a machine learning process to analyze SERS spectra data and determine antibiotic susceptible and/or resistant bacterial strains.

Previous work in AST involved acquiring clinical samples from patients, culturing the samples 24 to 72 hours, and using disk-diffusion or broth microdilution assays (among other techniques) to identify a suitable antibiotic for treatment. (See, e.g. Pulido, M. R., et al., J. Antimicrob. Chemother., 2013, 68, 12, 2710-2717; and Reller, L. B., et al., Clin. Infect. Dis., 2009, 49, 11, 1749-1755, the disclosures of which are incorporated herein by reference). Yet AST results are required within 30 to 60 minutes for reducing inappropriate use of antibiotics and optimal patient outcomes. (See, e.g. Schoepp, N. G., et al., Sci. Transl. Med., 2017, 9, 410, eaal3693; the disclosure of which is incorporated herein by reference). Point-of-care genomic AST, using genetic markers (genes, plasm ids or mutations) associated with AMR, potentially obviates the need for culturing and has shown results on the time scale of hours. Yet the presence of resistance genes does not necessarily translate to expressed (phenotypic) resistance. (See, e.g. Baltekin, Ö., et al., Proc. Natl. Acad. Sci., 2017, 114, 34, 9170-9175; the disclosure of which is incorporated herein by reference). Furthermore, genotypic AST detects only known genes and mutations associated with resistance, and does not allow for guarding against the emergence of newly evolved resistance mechanisms. For at least these reasons, phenotypic AST can be a gold standard and often genomic AST may still require phenotypic validation.

Metabolomic analyses of the bacterial response to antibiotic treatment, using standard methods such as mass spectrometry, show that the mechanism of killing of antibiotics generally depends on the dysregulation of core metabolic function and substantial changes in metabolite profiles occur within 30 minutes after antibiotic exposure. (See, e.g. Jung, J. S., et al., J. Clin. Microbiol., 2016, 54, 11, 2820-2824; and Belenky, P., et al., Cell Rep., 2015, 13, 5, 968-980; and Rowan, A. D., et al., Microb. Cell, 2016, 3, 4, 178-180; the disclosures of which are incorporated herein by reference). In order to reduce the time required for phenotypic AST, a metabolomics approach rather than direct measurement of cell growth or viability can be adapted as recent studies on metabolite responses to antibiotic exposure indicate that a rapid metabolic profiling technique is able to detect phenotypic susceptibility or resistance to antibiotics. (See, e.g. Rowan, A. D., et al., Microb. Cell, 2016, 3, 4, 178-180; the disclosure of which is incorporated herein by reference). Yet this approach can be time consuming: metabolomics approaches introduce an enormous parameter space. For example, the E. coli metabolome contains over 2600 different metabolites.

Machine learning (ML), especially deep learning, has emerged as a promising force to improve healthcare, with ML approaches surpassing the performance of doctors in computer vision tasks like diagnosing skin and breast cancer. (See, e.g. Esteva, A., et al., Nature, 2017, 542, 7639, 115-118; and Cheng, J. Z., et al., Sci. Rep., 2016, 6, 24454, the disclosures of which are incorporated herein by reference). Raman spectroscopy together with ML has shown promise for AST and reports that analysis can benefit from enhancements in SERS when coupled with ML. (See, e.g. Ho, C. S., et al., Nat. Commun., 2019, 10, 1, 1-8; the disclosure of which is incorporated herein by reference). Since the use of principal component analysis to demonstrate single molecule detection, progress has been made in applying ML techniques to solve SERS problems, for example, fully-connected artificial neural networks for analyte concentration regression, DNA classification, and cancer detection, convolutional neural networks (CNNs) for classification of metabolite signals support vector machines for classification of drug use from urine, and genetic algorithms for cancer diagnoses. (See, e.g. Le Ru, E. C. et al., J. Phys. Chem. B, 2006, 110, 4, 1944-1948; and Li, X. et al., Opt. Express, 2015, 23, 14, 18361-18372; the disclosures of which are incorporated herein by reference). It may seem like an ML based SERS approach to AST would be easily implemented due to the practicality of collecting large SERS datasets of bacterial metabolites. Yet, in addition to the challenge of fabricating SERS sensors with reproducible response, one key challenge which faces this approach as implemented in prior SERS AST is rapid AST sensor data need to be validated with traditional AST approaches to be accepted into practical use. This means that every AMR status label that corresponds to a SERS spectrum can require a 24 hour to 72 hour culturing process. Considering that deep ML algorithms that tackle healthcare problems may require thousands of labeled examples, this timeline can represent an enormous barrier for the development of SERS AST in a clinical setting. (See, e.g. Lussier, F. et al., ACS Nano, 2019, 13, 2, 1403-1411; Nam, J. M. et al., Acc. Chem. Res., 2016, 49, 12, 2746-2755; and Belkum, A. van et al., J. Clin. Microbiol., 2013, 51, 7, 2018-2024; the disclosures of which are incorporated herein by reference).

SERS platforms detecting bacterial metabolite profiles and correlating antibiotic susceptibility in accordance with several embodiments of the invention can improve efficiency and sensitivity in phenotypic AST. Many embodiments access differences in bacterial metabolites as a sensing platform for diverse analytes detection. Bacterial cells can be used as a signal amplification platform for sensing applications combined with machine learning data analysis processes in accordance with several embodiments. Antibiotics that are effective against a given strain of bacteria can induce large changes in metabolite profiles of the cells. Some embodiments detect these changes in the metabolite profiles with sensitivity and accuracy to determine antibiotic effectiveness. In a number of embodiments, analytes that may affect bacterial metabolism including (but not limited to) toxic metals and pesticides, can be detected by analyzing bacterial metabolite changes.

Many embodiments implement signal amplification component including (but not limited to) bacterial cells and efficient data analysis processes including (but not limited to) machine learning in sensing applications. Several embodiments enable sensing of arsenic and chromium down to concentrations relevant to environmental water quality testing. The sensing platform may constitute a rapid, portable, and low-cost alternative to conventional analytical instrumentation for water quality monitoring, such as mass spectrometry, while not sacrificing much in sensitivity. In certain embodiments, the sensing platform can be applied to contaminants of interest in water quality monitoring including (but not limited to) endocrine disruptors, pesticides and herbicides, and per- and polyfluoroalkyl substances (PFAS). Several embodiments provide the sensing platform can be used to sense differences in nutrient conditions of bacterial growth media.

SERS sensing platforms using bacterial metabolite profiles and correlating antibiotic susceptibility in accordance with several embodiments of the invention can improve efficiency and sensitivity in phenotypic AST. In several embodiments, SERS sensors can be used to acquire input data for rapid AST. SERS sensors can detect small molecules with a label-free approach in accordance with various embodiments. Some embodiments provide that SERS sensors exhibit high sensitivity with detection concentrations at about 1 part per trillion. Integrated with nanomanufacturing methods, such as the 2-dimensional physically activated chemical (2PAC) assembly method, SERS sensors can have controlled plasmonic nanogaps on their substrates and permit the rapid acquisition of large datasets. Many embodiments provide that SERS sensors are able to rapidly acquire large datasets due to their high sensitivity and consequently short exposure times to acquire reproducible spectra. In some embodiments, 2PAC fabricated SERS sensors are able to detect single molecule, and quantify molecular concentration down to about 10 fM. Several embodiments implement portable and cost effective spectrometers in the sensing platform.

Machine learning assisted analysis of AST spectra may be able to capture the diversity and complexity of the bacterial systems observed in a clinical setting in a robust manner. In many embodiments, a machine learning process can be implemented to reduce the amount of labeled data for SERS spectra analysis to achieve fast and robust AST. Several embodiments provide that the machine learning processes utilize models that are trained using SERS spectra as input datasets. Many embodiments generate antimicrobial susceptibility as outputs based on the latent space between the input SERS spectra and the properties that are learned during the training of the machine learning model. In some embodiments, the output properties can include (but are not limited to): antibiotic susceptible metabolite profile, antibiotic resistance metabolite profile, antibiotic temporal response, and antibiotic dosage response. In some embodiments, a machine learning model training process can be unsupervised, semi-supervised, supervised, and combinations thereof. Examples of machine learning models include (but are not limited to): variational autoencoder (VAE), support vector machine (SVM), convolutional neural networks (CNNs), and Bayesian Gaussian mixture. In several embodiments, data informed transfer learning can be leveraged to improve predictive machine learning models of complex metabolite response present in SERS spectra. Classification accuracy can reach around 99.3% in accordance with some embodiments of the invention.

Some embodiments include SERS sensors with machine learning processes that can generate an antibiotic sensitivity response in less than one hour. In several embodiments, the response time is less than 40 minutes. Further embodiments include machine learning analysis of SERS spectra capable of identifying bacterial response to antibiotics in dosages up to 10-fold lower than the minimum inhibitory concentration as determined in cell growth assays.

In many embodiments, SERS analysis of cell lysate from Pseudomonas aeruginosa (P. aeruginosa) that has been treated with different antibiotics of varying efficacy can be used to train algorithms to predict antibiotic resistance or susceptibility. The VAE, a deep generative model, can produce good clustering behavior of SERS spectra from different antibiotic treatments into its latent space in accordance with various embodiments. Several embodiments can extend such method to investigate the dose and temporal response of P. aeruginosa and Escherichia coli (E. coli), where differentiation may begin at around 20 minutes for E. coli and around 40 minutes for P. aeruginosa and when exposed to antibiotic concentrations 10 fold lower than reported values of the MIC for both pathogens. In further embodiments, VAE's ability to capture the data distribution may allow for interpretation of spectral features from each SERS cluster in the latent space that can then be used to guide researchers' gathering of unlabeled data. Training algorithms with 63 targeted mixtures of bacterial metabolites in vibrational regions of interest revealed by the VAE can improve clustering of SERS AST spectra without any increase of labeled data in some embodiments. Many embodiments implement an unsupervised Bayesian Gaussian Mixture model to achieve about 99.3% accuracy on a test AST dataset, which is higher than a deep CNN transfer learning based approach for fewer than 10 example spectra.

Methods and systems for integrating machine learning analysis with SERS nanosensor platform that can be utilized in rapid AST in accordance with various embodiments of the invention are discussed further below.

### Rapid Antimicrobial Susceptibility Testing Process

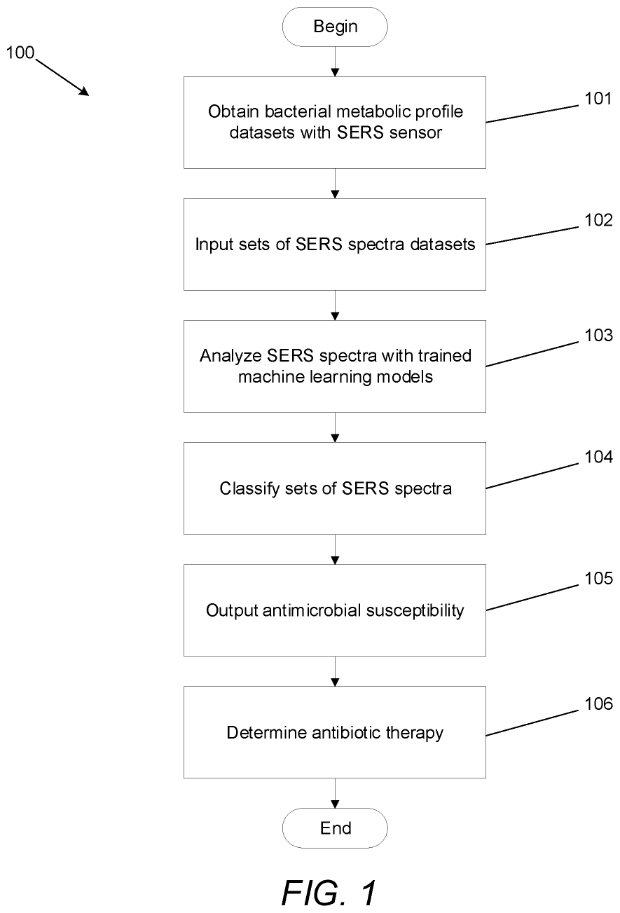

Many embodiments utilize accurate and transferable machine learning processes to predict properties including (but not limited to) antimicrobial susceptibility based on input features using computations including (but not limited to) a variational autoencoder (VAE). A method for implementing rapid antimicrobial susceptibility test using a machine learning process in accordance with an embodiment of the invention is illustrated in FIG. 1. The process 100 can begin by obtaining a bacterial metabolic profile dataset (101) with SERS sensors. Some embodiments include obtaining bacterial metabolic profile datasets from pure bacterial cultures. Examples of pure bacterial cultures include (but are not limited to): Pseudomonas aeruginosa (P. aeruginosa), Escherichia coli (E. coli), uropathogenic E. coli, Enterococcus faecalis (E. faecalis), and Klebsiella pneumoniae (K. pneumoniae). In a number of embodiments, bacterial metabolic profile datasets can be obtained from defined bacterial co-cultures. Examples of defined bacterial co-cultures include (but are not limited to): co-cultures of E. coli, K. pneumoniae, and P. aeruginosa, co-cultures of E. coli and Salmonella enterica serovar Typhimurium (S. typhimurium), pairwise co-cultures of uropathogenic E. coli, E. faecalis, and K. pneumoniae and co-cultures of all three treated or untreated with antibiotics. In certain embodiments, bacterial metabolic profile datasets can be obtained from clinically relevant bacteria strains including (but not limited to) uropathogenic strain of E. coli. In several embodiments, bacterial metabolic profile can be obtained post antibiotic exposure and correlating with antibiotic susceptibility. As can readily be appreciated, any of a variety of bacterial metabolic profile can be utilized as appropriate to the requirements of specific applications in accordance with various embodiments of the invention.

Sets of SERS spectra for the input datasets can be obtained based on bacterial metabolic profile (102). In some embodiments, the SERS spectra can be pre-processed. In several embodiments, the SERS spectra are processed in three steps including smoothing, background subtraction, and scaling. As can readily be appreciated, any of a variety of input SERS spectra can be utilized as appropriate to the requirements of specific applications.

In certain embodiments, SERS spectra analysis are performed using machine learning processes (103). In a number of embodiments, the spectra analysis can be implemented with VAE. In several embodiments, the spectra analysis are performed with convolutional neural networks, semi-supervised VAE and SVM models. In some embodiments, the spectra analysis are implemented with unsupervised VAE and Bayesian Gaussian mixture models. Machine learning processes can be trained with SERS spectra of the input datasets.

During a training process (not shown) machine learning analysis can learn relationships between SERS spectra and clustering in latent space using a training dataset. In some embodiments, the training datasets can be labeled bacterial metabolic profile SERS spectra by using growth assays to generate labels for response of bacteria to different antibiotic exposure conditions. In several embodiments, the training datasets can be unlabeled metabolic profile SERS spectra. As can readily be appreciated, any of a variety of training datasets can be utilized as appropriate to the requirements of specific applications in accordance with various embodiments of the invention.

The machine learning analysis can utilize a trained model that describes latent space between the input SERS spectra and the properties that are learned during the training to perform a categorization and/or ranking (104) of antibiotic susceptibility in the input SERS spectra dataset. In many embodiments, the machine learning analysis can also identify antibiotic dosage response and/or temporal response that are not in the input dataset based upon regions of the latent space that contain spectra that the model predicts will have desirable properties. The various ways in which machine learning analysis can be utilized to identify molecular systems having desirable properties in accordance with various embodiments of the invention including specific examples are discussed further below.

In many embodiments, the machine learning analysis processes generate output datasets of antimicrobial susceptibility (105). The output antimicrobial susceptibility properties can include (but are not limited to): antibiotic susceptible metabolite profile, antibiotic resistance metabolite profile, antibiotic temporal response, and antibiotic dosage response. As can readily be appreciated, the specific features used as molecular system properties are largely only limited by the requirements of specific applications. Based on the output datasets, antibiotic therapy in response to antimicrobial susceptibility can be identified and developed (106).

While various processes for developing antibiotic therapy using rapid AST processes are described above with reference to FIG. 1, any of a variety of processes that utilize machine learning to analyze the SERS spectra can be utilized in the identification and/or development of antibiotic therapy as appropriate to the requirements of specific applications in accordance with various embodiments of the invention. Processes for obtaining SERS spectra and analyzing with machine learning models in accordance with various embodiments of the invention are discussed further below.

### Surface Enhanced Raman Scattering Sensors

In many embodiments, rapid AST incorporates a sensing platform with surface enhanced Raman scattering (SERS) sensors. Several embodiments describe that multiplex sensing and analyte quantitation with SERS sensors might be able to sense signals associated with shifts in central metabolic function.

SERS sensors can be sensitive at concentrations where analyte molecules are non-uniformly distributed across the surface in a complex background in accordance with various embodiments. These SERS sensors can provide a spectral fingerprint of samples using carefully designed nanoarchitectures to enhance otherwise undetectable signals of light scattered from molecular vibrations near gaps between nanospheres. U.S. patent application Pub. No. 2019/0064074 A1 to Ragan et al., describes SERS sensors with nanoarchitectures comprised of subwavelength metal nanosphere oligomers with uniform narrow gap spacings for plasmonic and metamaterial devices. A biosensor system based on such nanoarchitectures that is able to detect pathogenic and/or other organisms such as bacteria is also described. The SERS sensors show control of sub-nm nanogaps in many embodiments. The chemical assembly method for SERS sensors fabrication described in the patent application is low-cost, scalable, and capable of reproducibly probing individual molecules over mm² areas. The disclosure of U.S. patent application Pub. No. 2019/0064074 A1 is herein incorporated by reference.

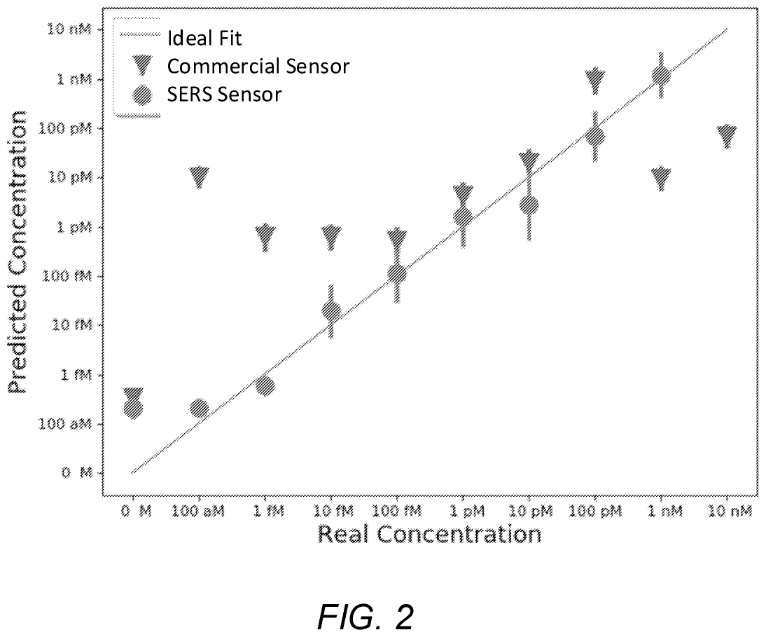

The data complexity of Raman spectra can be tackled by using nonlinear machine learning (ML) methods for SERS analysis. A machine learning analysis of Rhodamine 800 spectra of a SERS sensor and a commercially available sensor in accordance with an embodiment of the invention is illustrated in FIG. 2. FIG. 2 shows an example of a CNN analysis that improves the detection limit of a SERS sensor to about 10 fM. An implemented CNN regression model trained on SERS data of Rhodamine 800 results in limits of detection (LOD) and quantification (LOQ) of about 10 fM (about 10 ng/mL to 5 ng/m L) with prediction accuracy (r² value) of about 0.96 over a dynamic range of 6 orders of magnitude. This LOD is orders of magnitude lower than commercial sensors composed of gold coated nanofingers from Silemco™. Silemco™ sensors do not have control of nanogap size and chemistry compared to SERS sensors, and are inaccurate below 10 nM. (See, e.g. Thrift, W. J., et al., Anal. Chem., 2019, 91, 13337; the disclosure of which is incorporated herein by reference).

Surface Enhanced Raman Scattering Spectra Analysis with Variational Autoencoder

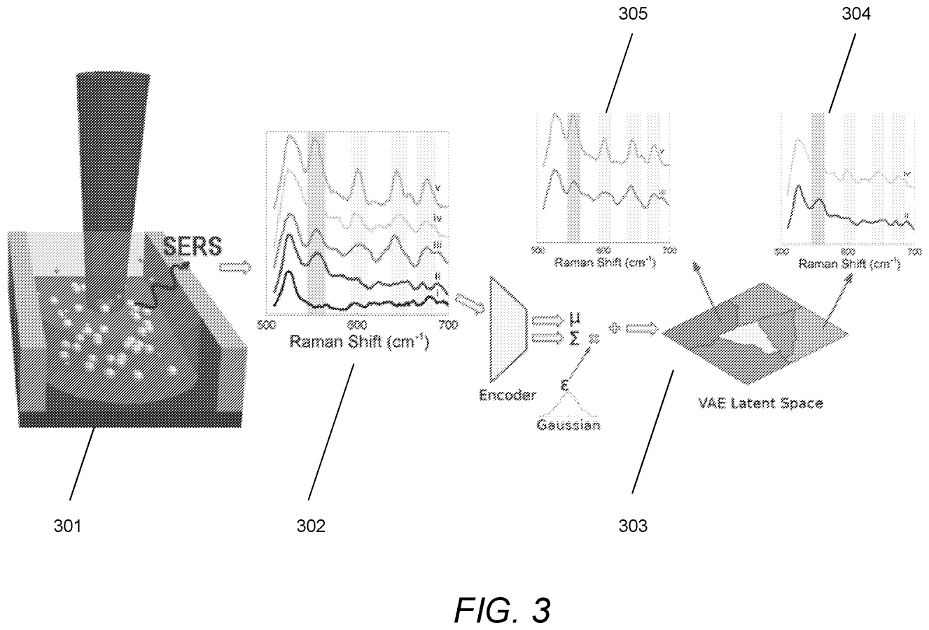

In many embodiments, machine learning assisted analysis of AST spectra are able to capture the diversity and complexity of the bacterial systems observed in a clinical setting in a robust manner. A sensing modality like the olfactory system can integrate the sensitive but non-selective detector signals of SERS and the complex signal processing capabilities of machine learning (ML) algorithms in accordance with many embodiments. A detection scheme for SERS data acquisition and machine learning analysis of bacterial metabolomic samples in accordance with an embodiment of the invention is illustrated in FIG. 3. Raman spectra (302) can be collected from cell lysate on highly sensitive SERS devices (301). A trained ML model can analyze the complex spectral information, aside from a few peaks assigned to known molecular vibrations, to recognize molecular signatures in complex mixtures in accordance with various embodiments of the invention. Machine learning algorithm (303) separates high dimensional training data into differentiable categories of resistant metabolite profile (304) corresponding to resistant cell populations and susceptible metabolite profile (305) corresponding to susceptible cell populations. Sample data can then be categorized with upwards of about 99% accuracy.

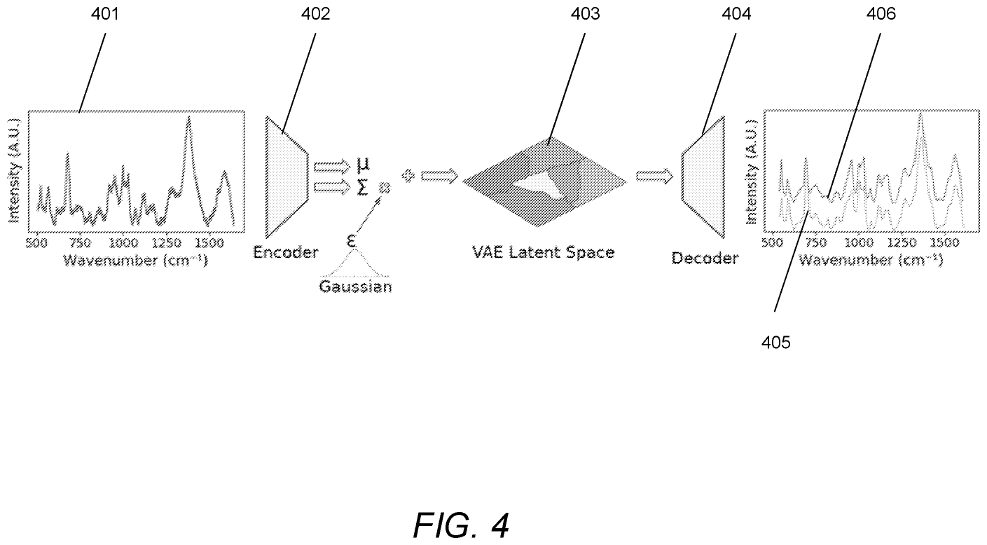

A trade-off may exist between the interpretability of ML models and their prediction accuracy when building models that capture the complexity of SERS spectra of bacterial metabolites. Several embodiments demonstrate that deep generative models including (but not limited to) the VAE, may help overcome this tradeoff by giving the user insight into the model's decision making. In many embodiments, the VAE can work by encoding a high dimensional data point (a SERS spectrum) into a low dimensional latent space to capture an essential representation of the data. In several embodiments, the VAE can be composed of an encoder network that encodes spectra as a Gaussian probability distribution in the 2-dimensional latent space, schematically depicted as μ and Σ, and a decoder network that takes points from the latent space and decodes them back into the original spectra. A scheme for using a VAE for SERS analysis in accordance with an embodiment is illustrated in FIG. 4. Plotted SERS spectra (401) can be used as training data. The SERS spectra can be encoded (402) in the VAE model into the latent space (403) as Gaussian distributions with mean μ and variance Σ. The encoder (401) and decoder (404) models can be deep convolutional neural networks. The spectrum (401) is encoded, decoded and plotted as the curve (405). The overlaid curve (406) highlights differences in spectra in clusters in VAE space.

Many embodiments provide that by encoding spectra as probability distributions in a low dimensional latent space, the VAE can provide 3 useful features: 1) Clustering, e.g., as all spectra are encoded as distributions, they can overlap with one-another. If overlapping distributions are not from similar spectra, the model can be heavily penalized during training. This can result in a well-structured latent space that enables the use of simple models to make predictions from encoded data. 2) De-noising, e.g., the low dimensional latent space does not contain enough information to encode for noise. This may improve predictions made from models trained on encoded data, especially for small amounts of labeled data. 3) Interpretation, e.g., encoding spectra as distributions enables that the latent space can be a continuous representation of the different classes of antibiotic treatments. Such features of VAE in accordance with embodiments may enable decoding spectra and visualizing variations across the latent space to ensure decoded spectra represents experimental data and identify vibrational features associated with susceptibility versus resistance.

### Variational Autoencoder Spectra Visualization and Semi-Supervised AST

In many embodiments, the VAE latent space can enable semi-supervised classification techniques for clustering of SERS spectra of bacterial lysates exposed to various antibiotic conditions. Several embodiments provide that this approach can reduce the time to acquire cell culture data, which is necessary to acquire labels including (but not limited to) antibiotic resistant, antibiotic susceptible. In some embodiments, SERS spectra of cell lysate from P. aeruginosa cultures are collected as a function of exposure time and type of antibiotic. In some embodiments, SERS spectra can be pre-processed prior to training the VAE.

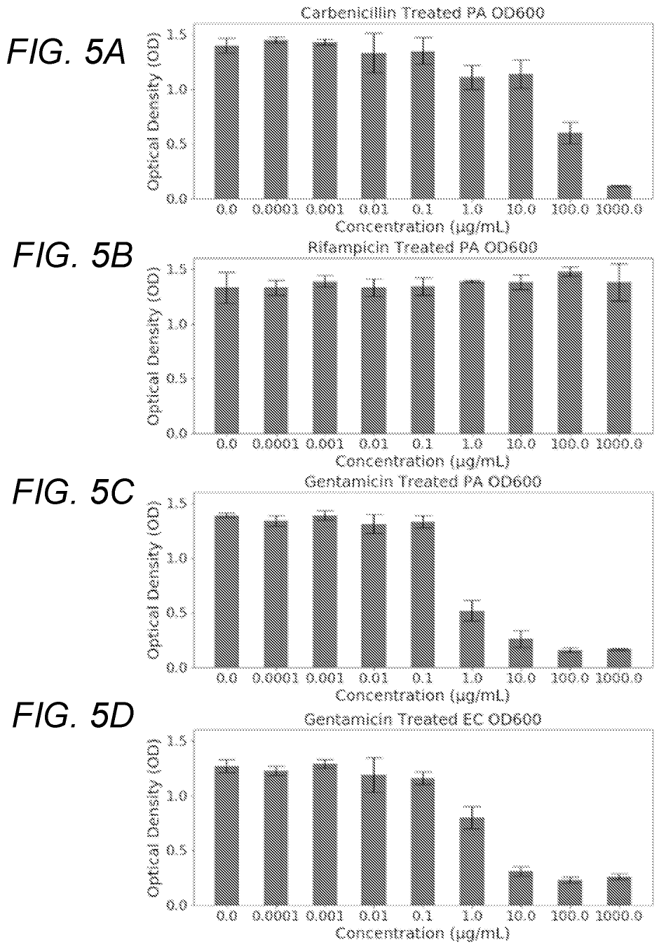

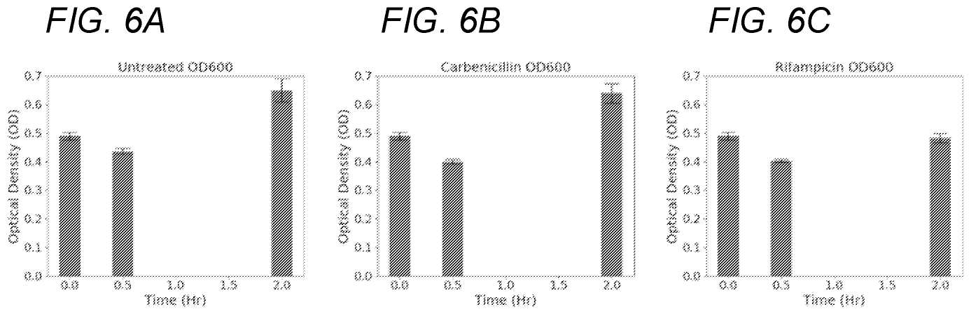

Optical density at 600 nm (OD₆₀₀) to identify the minimum inhibitory concentration (MIC) of A) carbenicillin, B) rifampicin, and C) gentamicin for Pseudomonas aeruginosa, and D) gentamicin for Escherichia coli in accordance with an embodiment is illustrated in FIGS. 5A-5D. The P. aeruginosa strain, PA14 used in some embodiments has differing susceptibility to carbenicillin [+C] and rifampicin [+R]. Several embodiments refer to the antibiotic treatments that are performed with 50 μg/mL and 400 μg/mL of carbenicillin and rifampicin, respectively as the AST dataset. These concentrations are just below the respective minimum inhibitory concentrations (MICs) for these antibiotics in P. aeruginosa, as shown in FIGS. 5A and 5B. OD₆₀₀ of a P. aeruginosa cell culture at indicated growth times after adjustment to OD at 0.5 at 0 h, A) without antibiotic treatment, B) with 50 μg/mL carbenicillin introduced at 0 h, and C) with 400 μg/mL rifampicin introduced at 0 h in accordance with an embodiment of the invention is illustrated in FIGS. 6A-6C. At 50 μg/mL and 400 μg/mL concentrations, P. aeruginosa does not exhibit any significant growth inhibition over the time of exposure of about 2 hour used for lysate preparations as shown in FIGS. 6A-6C.

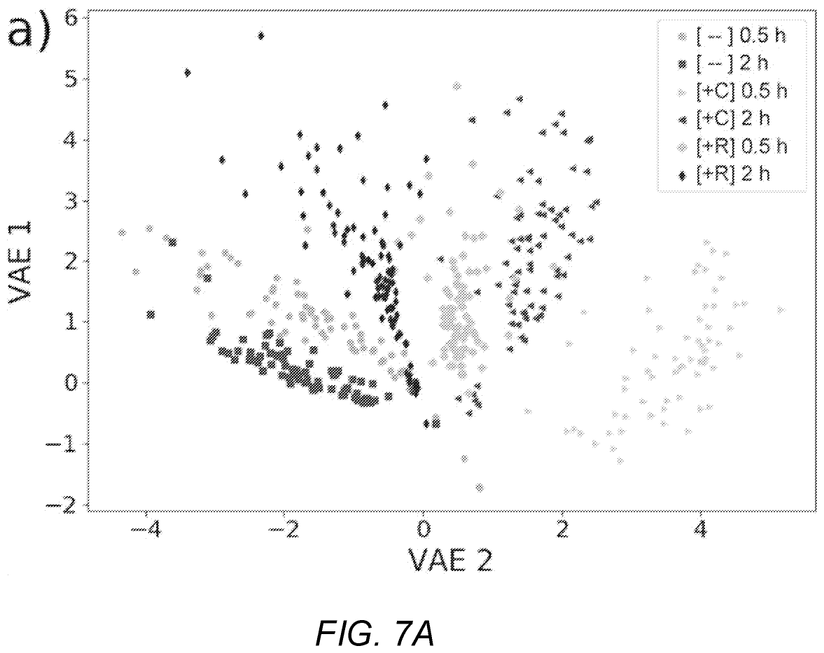

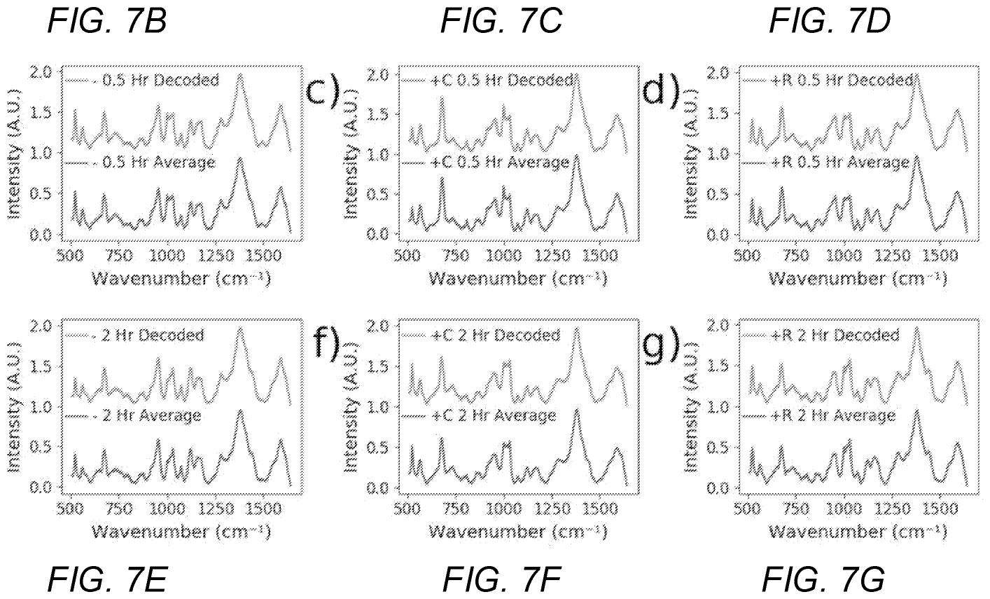

The VAE space from analysis of the AST dataset in accordance with many embodiments provide clustering of the different treatment classes. VAE space of AST spectra from untreated [−−] P. aeruginosa lysate (0.5 h/2 h), 50 μg/mL carbenicillin treated [+C] P. aeruginosa lysate (0.5 h/2 h), 400 μg/mL rifampicin treated [+R] P. aeruginosa lysate (0.5 h/2 h) in accordance with an embodiment of the invention is illustrated in FIG. 7A. There is a observable trend of cell viability across VAE 2, with small VAE 2 values corresponding to untreated bacteria [−−], intermediate VAE 2 values corresponding to treatment with rifampicin [+R], and large VAE 2 values corresponding to treatment with carbenicillin [+C]. This trend tracks with the expected efficacy of each treatment, with rifampicin being intermediate between the control and carbenicillin treatment due to the evident resistance of P. aeruginosa to gentamicin and rifampicin shown in FIGS. 5A-5D. Several embodiments provide that the appearance of this trend in the VAE space can be notable, highlighting the ability of the SERS sensors to successfully differentiate these samples; particularly since all antibiotic treatments are below their respective MIC and hence do not inhibit growth over the time scale of exposure (FIGS. 6A-6C). Some embodiments provide that larger relative VAE 1 values can be observed for 30 minute treatment times for all treatment classes as compared to 2 hour exposure. This is consistent with OD₆₀₀ measurements indicating cell recovery after 2 hour in FIGS. 6A-6C.

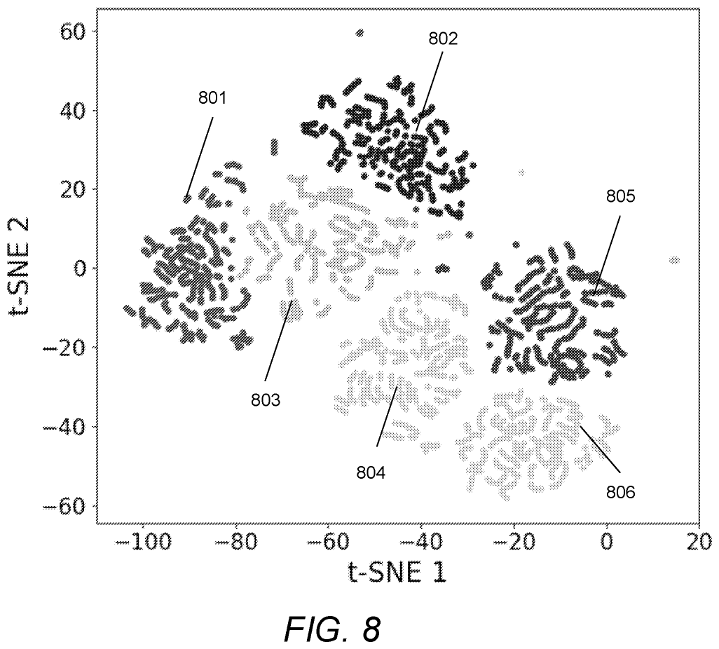

FIG. 8 illustrates a t-stochastic neighbor embedding (t-SNE) visualization of the spectra used to build the VAE latent spaces in accordance with an embodiment of the invention. 803 shows untreated [−−] P. aeruginosa lysate 0.5 hour. 801 shows untreated [−−] P. aeruginosa lysate 2 hour. 806 shows 50 μg/mL carbenicillin treated [+C] P. aeruginosa lysate 0.5 hour. 805 shows 50 μg/mL carbenicillin treated [+C] P. aeruginosa lysate 2 hour. 804 shows 400 μg/mL rifampicin treated [+R] P. aeruginosa lysate 0.5 hour. 802 shows 400 μg/mL rifampicin treated [+R] P. aeruginosa lysate 2 hour. As an unsupervised model, t-SNE can demonstrate that there are differences in the SERS spectra, which are discernable in the absence of user identification of labels. t-SNE is a visualization technique that can produce better clustering than VAE. Yet, despite the utility of t-SNE as a visualization tool, it is probabilistic in nature and its embeddings may not be used to develop a predictive model.

In several embodiments, the generative aspect of the VAE may allow for comparison of VAE generated spectra to averaged SERS spectra. FIGS. 7B-7G illustrate averaged SERS spectra from each treatment class and VAE generated spectra from the center of the class centroid in accordance with an embodiment where FIG. 7B depicts untreated [−−] P. aeruginosa lysate 0.5 hour; FIG. 7C depicts 50 μg/mL carbenicillin treated [+C] P. aeruginosa lysate 0.5 hour; FIG. 7D depicts 400 μg/mL rifampicin treated [+R] P. aeruginosa lysate 0.5 hour; FIG. 7E depicts untreated [−−] P. aeruginosa lysate 2 hour; FIG. 7F depicts 50 μg/mL carbenicillin treated [+C] P. aeruginosa lysate 2 hour; and FIG. 7G depicts 400 μg/mL rifampicin treated [+R] P. aeruginosa lysate 2 hour. The similarity between generated and experimental spectra in accordance with such embodiments provide that the clustering behavior reflects trends in the experimental data. Additionally, while the SERS spectra is preprocessed, the VAE can de-noise the spectra further. This can be supported as the VAE generated spectra are almost indistinguishable from the averages of experimental spectra in some embodiments.

Antimicrobial Susceptibility Testing Response with Variational Autoencoder

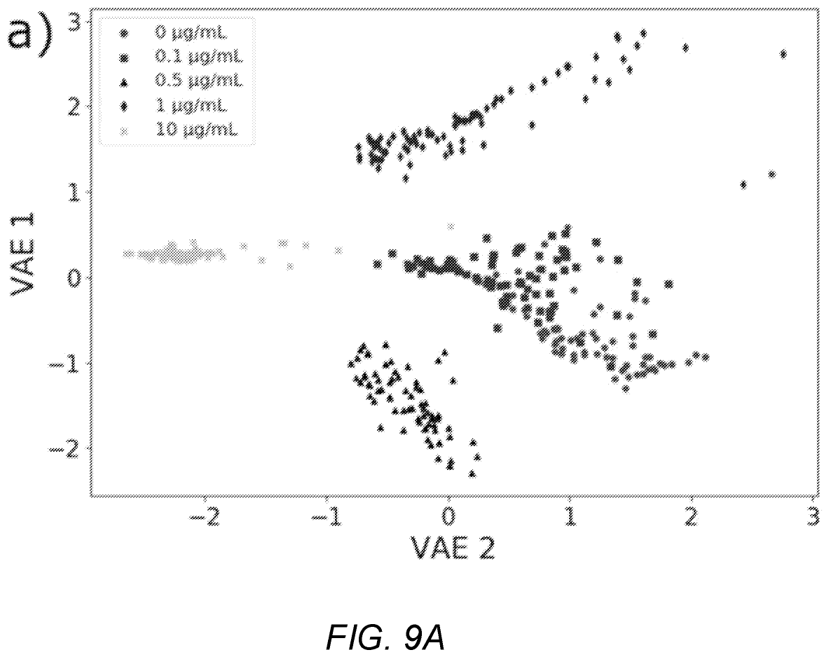

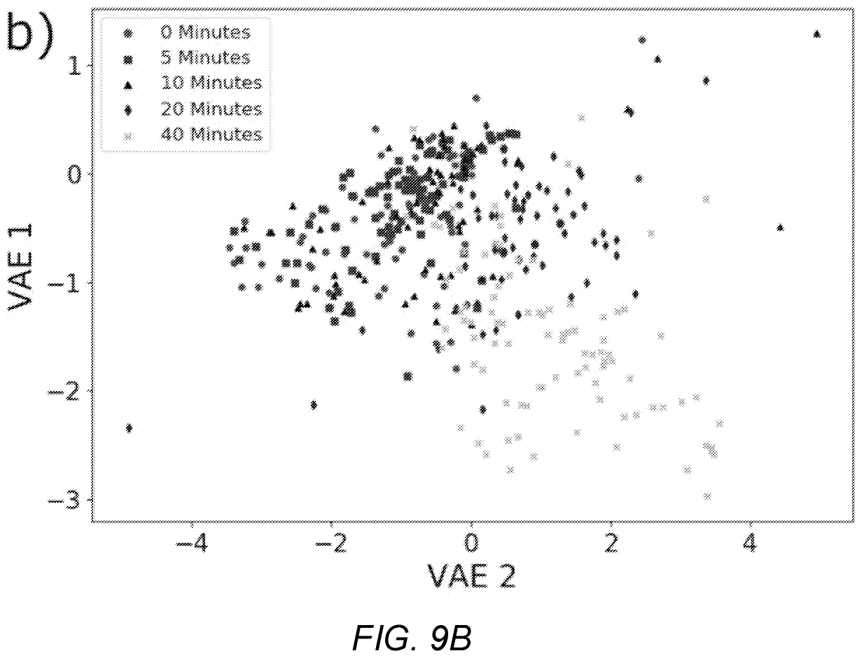

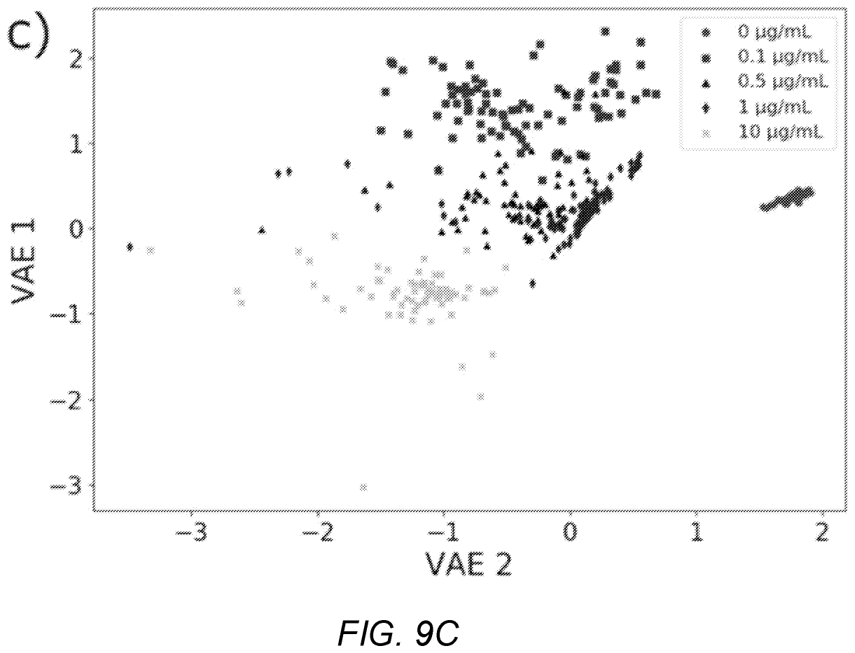

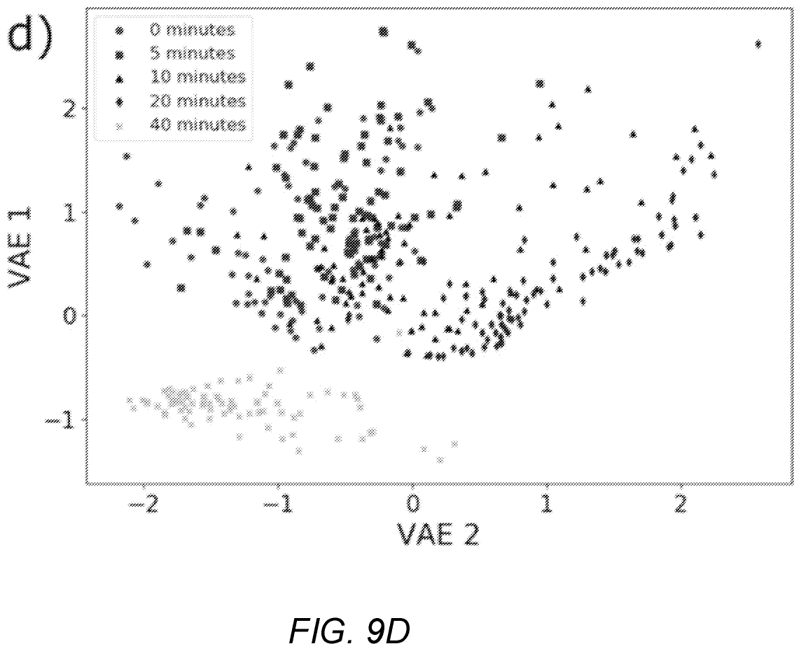

Many embodiments provide VAE performance on differentiating spectra from multiple bacterial species, a different antibiotic, time points, and dosages. SERS spectra from cellular lysate with varying dose and temporal response of P. aeruginosa and E. coli when exposed to gentamicin are analyzed similarly as the AST dataset in several embodiments. When analyzing the SERS spectra from E. coli and P. aeruginosa with the VAE method, differentiation of P. aeruginosa lysate data at concentrations at about 0.1 μg/mL, an order of magnitude below the MIC, can be observed in accordance with some embodiments. FIG. 9A illustrates VAE latent space analysis of SERS spectra of dose response of P. aeruginosa with analysis of 0, 0.1, 0.5, 1, and 10 μg/mL gentamicin dosed lysate respectively in accordance with an embodiment. In several embodiments, lysate from P. aeruginosa exposed to 10 μg/mL gentamicin (10×MIC) can be analyzed with VAE method and differentiated from control data in the VAE space at about 20 minute after initial exposure. The short temporal response time in accordance with some embodiments is much earlier than reported 2 hour response time for SERS AST. FIG. 9B illustrates VAE latent spaces of temporal response of P. aeruginosa with lysate processed after 0, 5, 10, 20, and 40 minutes of 10 μg/mL gentamicin dosage respectively in accordance with an embodiment. In further embodiments, E. coli lysate data can be differentiated from control treatment even at 0.1 μg/mL. FIG. 9C illustrates VAE latent space analysis of SERS spectra of dose response of E. coli with analysis of 0, 0.1, 0.5, 1, and 10 μg/mL gentamicin dosed lysate respectively in accordance with an embodiment of the invention. In several embodiments, the temporal response of E. coli lysate data shows earlier differentiation than P. aeruginosa, showing discernable clustering at about 10 minute after initial exposure to gentamicin, and increasingly segregated clusters are evident at 20 and 40 min time points. FIG. 9D illustrates VAE latent spaces of temporal response of E. coli with lysate processed after 0, 5, 10, 20, and 40 minutes of 10 μg/mL gentamicin dosage respectively in accordance with an embodiment.

From a ML perspective, conventional growth assays can be considered as a means of generating labels for response of bacteria to different antibiotic exposure conditions, and the SERS spectra as the resultant labeled data. The mismatch between the time for label generation and data generation might motivate investigating semi-supervised machine learning approaches. The correspondence between the VAE encoded SERS spectra and the antibiotic treatment classes as shown in FIGS. 7A-7G in accordance with embodiments indicates a semi-supervised approach using the VAE latent space.

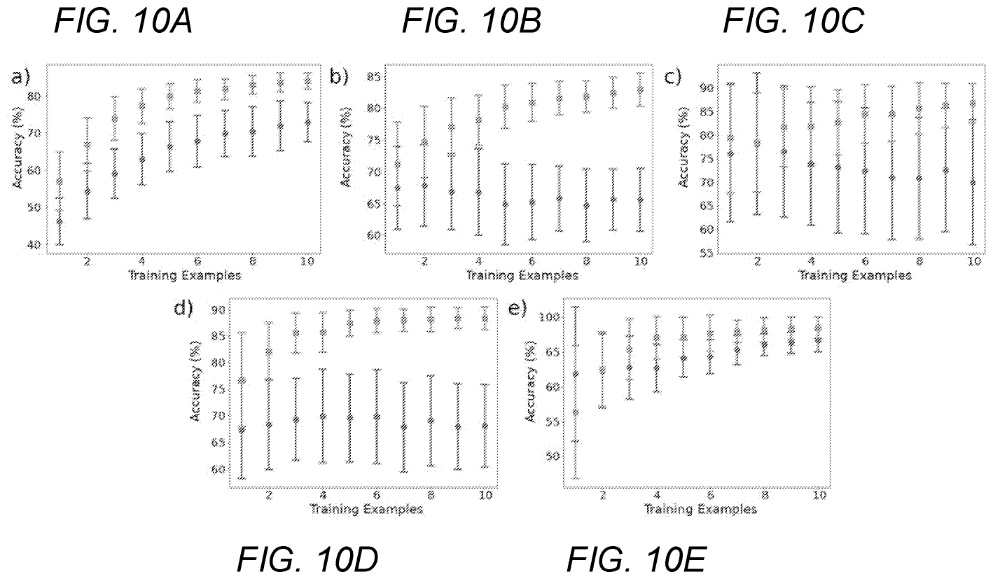

Many embodiments provide training of a support vector machine (SVM) model on preprocessed spectra and VAE encoded spectra as a function of the number of training examples. SVMs are chosen as discriminative model due to their resistance to overfitting. In some embodiments, SVM model fitting may not have as high an accuracy as a convolutional neural network but the resistance to overfitting can support whether the predictions are correlated with antibiotic susceptibility or not. Several embodiments provide the classification accuracy of the SVM model of preprocessed and VAE encoded spectra for both the AST dataset as well as the E. coli and P. aeruginosa dosage and temporal response datasets. FIGS. 10A-10E illustrate SVM model performance versus the number of training examples from the AST dataset (P. aeruginosa untreated and treated with carbenicillin or rifampicin) analyzed in accordance with an embodiment. SVM models are evaluated on preprocessed spectra (SVM), and on VAE encoded spectra (VAE SVM). The datasets used are: FIG. 10A—AST dataset, FIG. 10B—P. aeruginosa dose response dataset, FIG. 10C—P. aeruginosa temporal response dataset, FIG. 10D—E. coli dose response dataset, and FIG. 10E—E. coli temporal response dataset. Temporal response datasets, FIG. 10C and FIG. 10E are performed with three classes, 0 minutes, 20 minutes, and 40 minutes, while dose response datasets have the full five classes, and the AST dataset has six classes. Lysate used to collect SERS spectra in FIG. 10B-10E are exposed to gentamicin. In various embodiments, SVM analysis of VAE encoded spectra can perform much better than analysis of preprocessed spectra. A classification accuracy in discriminating between the six antibiotic treatment conditions of the AST dataset of about 83.7±2.6% can be achieved for the former case with approximately 10 labeled samples generated from growth assays in accordance with some embodiments. This is compared to a performance of 72.9±5.2% with a SVM trained on spectra that have not been encoded. The performance increase of SVM due to VAE encoding is more pronounced on dose and temporal response datasets.

The SVM analyses in accordance with many embodiments provide that a predictive SERS AST model with relatively high accuracy can be achieved with 10 or fewer labeled samples generated from growth assays, representing significant time and cost savings for clinical implementation of SERS AST.

### Data Informed Transfer Learning for Rapid Antimicrobial Susceptibility Testing

Many embodiments implement training models from unlabeled data. In various embodiments, models can be trained with metabolites in water without the time consuming step of culturing bacteria. Traditional transfer learning with deep neural networks can be done by training a model including (but not limited to) a convolutional neural network (CNN) with the large dataset, and then fine tuning the model's parameters with the smaller dataset to improve model predictions such as classification accuracy. In several embodiments, the use of a generative ML method may enable an informed approach to transfer learning. The high interpretability of the VAE generated SERS spectra may identify the useful vibrational information and correspondingly target additional data collection to improve classification accuracy.

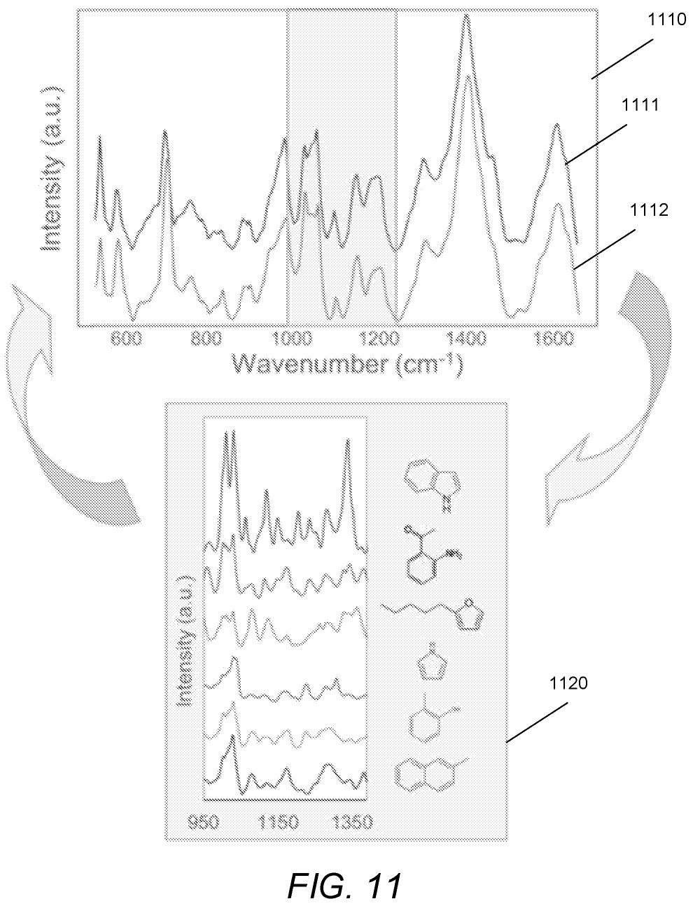

Some embodiments implement sampling 100 VAE generated spectra between the average response of the 0.5 hour carbenicillin-treated lysate data and that of the untreated 2 hour lysate data to visualize spectral features shift as a result of antibiotic exposure and ensuing changes in metabolites. In such embodiments, the progression shows that the bacterial lysate response to antibiotics can be most associated with changes in the 1100 cm⁻¹-1200 cm⁻¹ bands. Vibrational features in this frequency range can be associated with aromatic functional groups. Several embodiments include 6 generic, volatile aromatic bacterial metabolites: 2-methyl napthalene, o-cresol, 2-amino acetophenone, pyrrole, 2-pentyl furan, and indole to construct a “metabolite mixture” dataset to use for data informed transfer learning. In some embodiments, the “metabolite mixture” dataset can be generated by collecting SERS spectra from 63 aqueous solution combinations of between 1 to all 6 of these metabolites at a total concentration of about 1 part per million. This approach of producing easy-to-collect spectra (large, metabolite mixture dataset) based on observations of difficult-to-collect spectra (24 hour cell culture AST dataset) can be beneficial of data informed transfer learning for SERS analysis in accordance with some embodiments. FIG. 11 illustrates the cycle of data informed transfer learning in accordance with an embodiment of the invention. 1110 shows VAE encoded spectra P. aeruginosa lysate 0.5 hour after being treated with carbenicillin (1111) and untreated (1112). 1120 shows SERS spectra (from bottom to top) of 2-methyl napthalene, o-cresol, pyrrole, 2-pentyl furan, 2-amino acetophenone, and indole at concentration of 1 ppm. The dataset in accordance with certain embodiments can have relevant vibrational modes, expand the VAE space and produce bigger differences between the encodings of the AST dataset, resulting in improved classification accuracy.

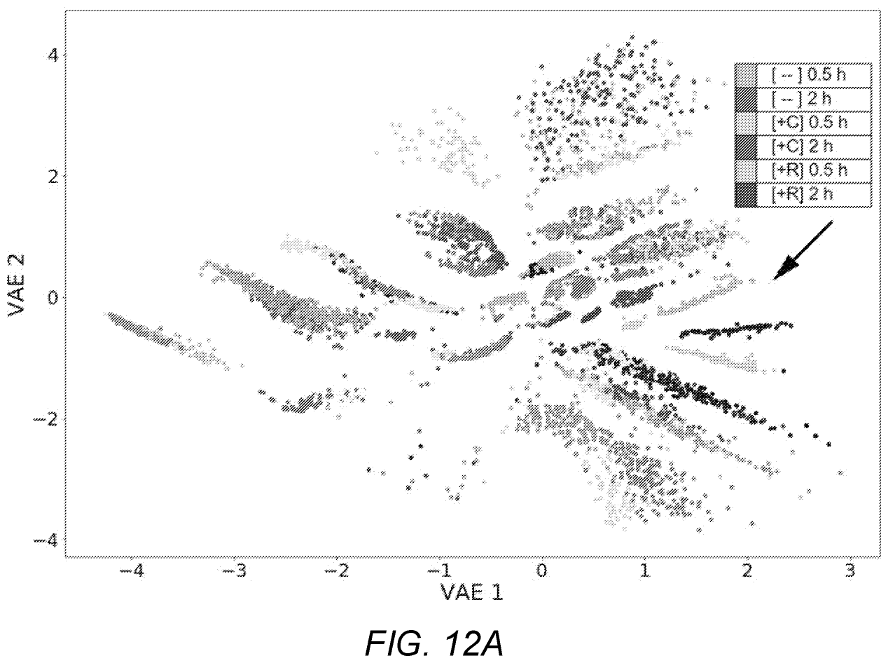

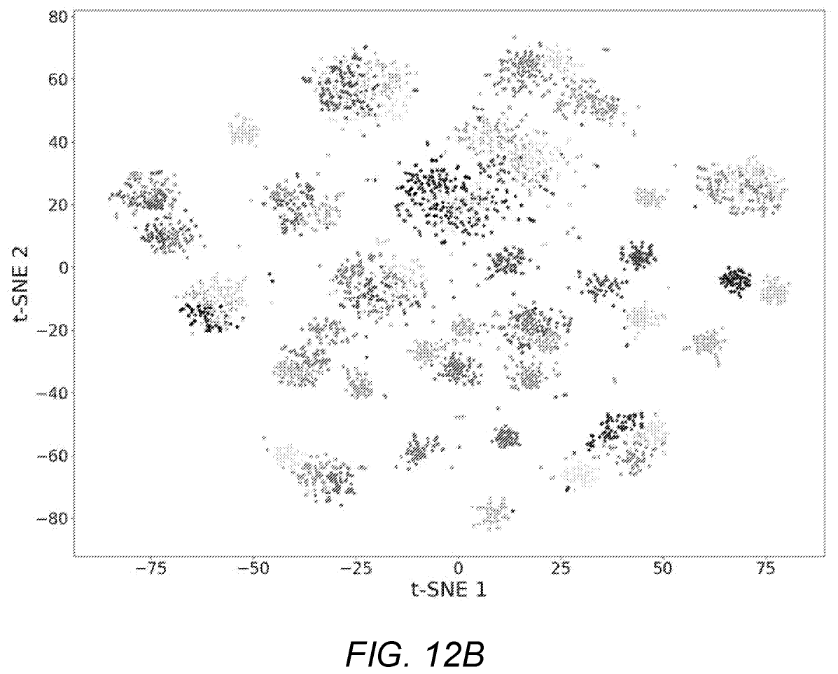

In several embodiments, the combined VAE latent space can be constrained to 2 dimensions so that it can be easily examined. The ‘combined’ dataset can benefit from a higher (32) dimensional latent space when visualizing clustering of different metabolite mixtures using t-SNE, which prioritizes preserving the neighboring distances of spectral data points in various embodiments. FIG. 12A illustrates VAE latent space from the test portion of the combined metabolite mixture and AST datasets in accordance with an embodiment. All 63 possible mixture combinations of metabolites 2-methyl napthalene (A), o-cresol (B), 2-amino acetophenone (C), pyrrole (D), 2-pentyl furan (E), and indole (F) are plotted with legend of color and corresponding mixture. FIG. 12B illustrates t-SNE visualization of a 32-dimensional metabolite combination VAE space in accordance with an embodiment of the invention. The improved clustering of the AST dataset, as shown in the center right side of FIG. 12A, is better than the clustering shown by the VAE model. Culture-free and easily acquired datasets of bacterial metabolites can be leveraged to improve predictive models of complex metabolite response present in bacterial lysate.

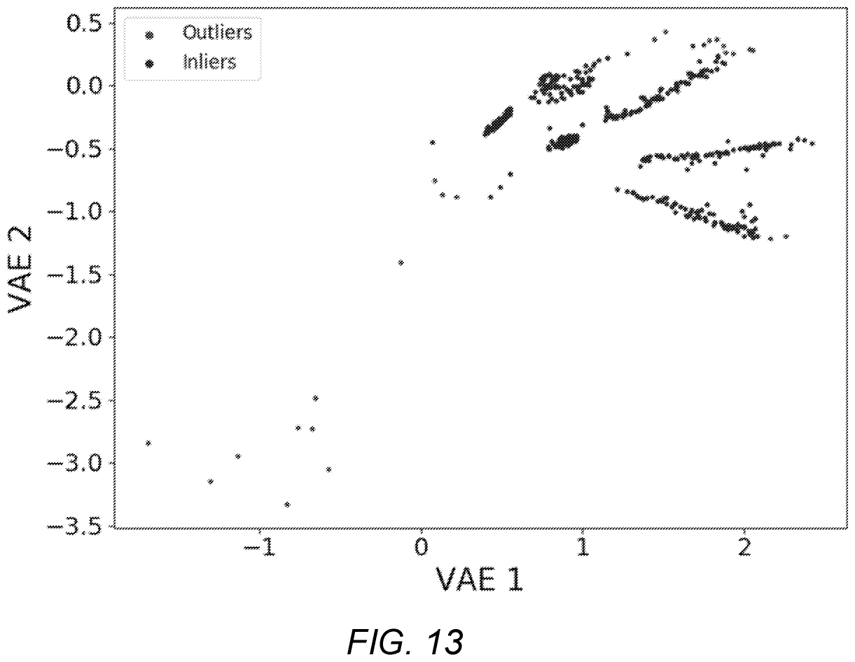

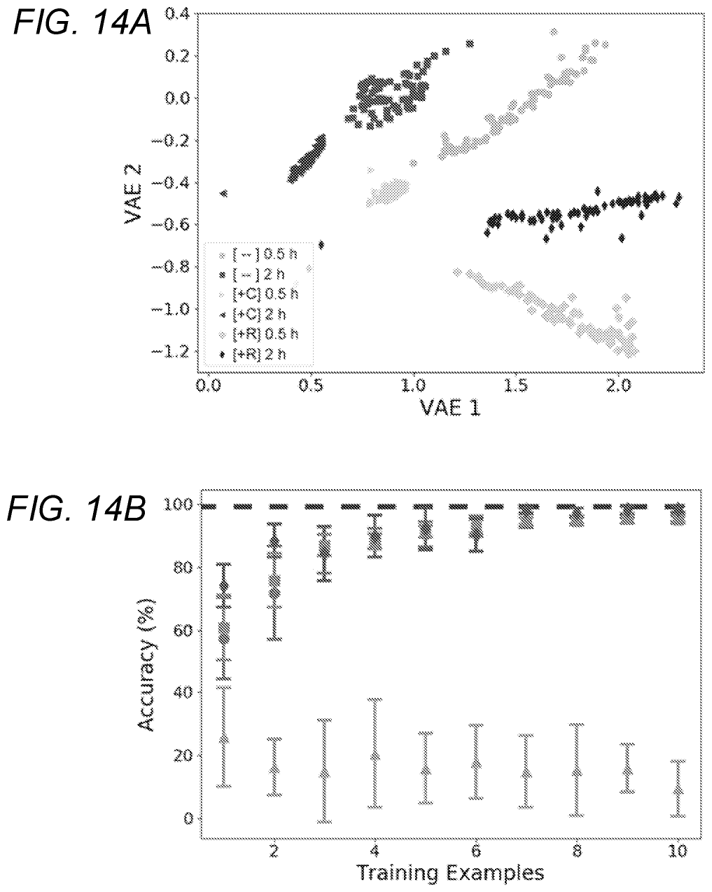

Many embodiments implement a predictive model. The VAE encoding show that outliers in the spectral data set can be identified. These spectra can be removed to improve accuracy with an isolation forest set to remove 5% of spectra. FIG. 13 illustrates isolation forest predictions of AST spectra that have been encoded with the combination VAE model in accordance with an embodiment of the invention. Several embodiments provide an unsupervised Bayesian Gaussian mixture model of the outlier removed AST dataset encoded with the combined VAE. FIG. 14A illustrates Bayesian Gaussian Mixture analysis of combined VAE encoded P. aeruginosa AST test spectra in accordance with an embodiment. Nearly perfect identification of the different classes can be achieved, with an accuracy of about 99.3%, with the only information given to the model is the number of clusters to expect (6). In several embodiments, the cycle can be repeated with the new VAE space, and more easy-to-collect unlabeled data if higher accuracy is needed. Some embodiments provide that the combined VAE encoding groups the different antibiotic conditions together, which is important for application of determining antibiotic susceptibility in large, clinical datasets.

Several embodiments compare data informed transfer learning approach with traditional transfer learning. In some embodiments, transfer learning is performed by taking the 6 unmixed metabolite datasets (e.g. 2-methyl naphthalene, o-cresol, 2-amino acetophenone, pyrrole, 2-pentyl furan, and indole dissolved in water by themselves), and training a model with those spectra. The weights of these trained networks are then fine-tuned with the few-example AST dataset.

FIG. 14B illustrates comparison of transfer learning model performance in accordance with an embodiment. Two models are compared, a multilayer perceptron (MLP), which is a fully connected artificial neural network with 1 hidden layer and a convolutional neural network (CNN), which is composed of 4 1D CNN layers. The MLP is trained with VAE encoded data and the CNN is trained with preprocessed SERS spectra without VAE encoding. Transferred model weights are trained using the unmixed preprocessed spectra from the metabolite dataset. These models are fine-tuned with examples from the AST dataset and evaluated on test spectra. Transferred model CNN (squares) and MLP (diamonds) are compared with the same architectures, CNN (circles) and MLP (triangles) without transfer learning using Xavier weight initialization as a function of the number of training examples. This procedure is performed 10 times for each example number. The resultant mean and standard deviation of the model accuracy are plotted in FIG. 14B as a function of the number of training examples. The unsupervised Bayesian Gaussian mixture method (dashed line) achieves the highest accuracy at 99.3%.

Several embodiments provide that CNNs can be a powerful tool for inference from SERS spectra. Even without transfer learning a CNN can achieve good results with 4 examples. Transfer learning with the CNN improves results regardless of the number of examples but the difference can be pronounced with very few examples. The transfer learned MLP (diamonds) is determined to produce good predictions for 1 shot learning with transferred learned CNN (squares) approaching similar performance as this MLP model for three examples. On the other hand, an MLP without transfer learning does not yield good predictions from the VAE space, likely due to underfitting as there are only 2 features. None of these models can outperform data informed transfer learning approach using the simple Bayesian Gaussian Mixture model.

### Materials and Methods

The following embodiments provide specific combinations of materials and methods that enable rapid AST with SERS sensors and machine learning analysis. It will be understood that the specific embodiments are provided for exemplary purposes and are not limiting to the overall scope of the disclosure, which must be considered in light of the entire specification, figures and claims.

### Fabrication of SERS Sensors

The fabrication process of SERS sensors includes first block copolymer templates for Au nanosphere assembly attachment are prepared. Random poly(styrene-b-methyl methacrylate) (PS-b-PMMA) block copolymer is spin-coated onto a hydrofluoric acid (HF)-cleaned, and then water rinsed Si (001) wafer or glass slides and annealed for 72 hours. The wafer is rinsed with toluene rinse and lamella forming PS-b-PMMA block copolymer is spin coated onto the wafer, which is annealed for another 72 hours. Next, PMMA regions within the block copolymer are selectively functionalized with amine terminated end groups by immersing a 1 cm×1 cm piece of the wafer in dimethyl sulfoxide (DMSO). This substrate is then transferred into an ethylenediamine/DMSO solution (5% v/v). Both immersions are performed for 5 min without rinsing between steps. The functionalized template is then rinsed with isopropyl alcohol (IPA) for 1 min and dried under nitrogen for immediate use.

An electrohydrodynamic flow driven assembly of Au nanospheres can be used to generate assemblies with the following method: Au nanosphere solution (0.1 mg/mL, 3 mL) is added to a clean 10 mL glass beaker. N-hydroxy sulfosuccinimide (s-NHS, 20 mM) in 2-(N-morpholino)ethanesulfonic acid (MES, 0.1 M) buffer (35 μL) is added to the nanosphere solution and swirled. Next, 1-ethyl-3-[3-(dimethylamino)propyl] carbodiimide hydrochloride (EDC, 8 mM) in MES (0.1 M) buffer (35 μL) is added to this solution and swirled. The solution is brought to, and maintained at 60° C. with a hot plate. The functionalized block copolymer-coated Si substrate is placed vertically into the solution and held in place with alligator clips, taking care to avoid any contact of the alligator clips with the solution. A 1 cm×1 cm Pt mesh is placed parallelly 1 mm away from the substrate. 1.2 V is applied across the mesh and substrate using a DC power supply for 10 min. Everything is then rinsed with IPA for 1 min and dried under nitrogen. The process is repeated with the same substrate and a fresh nanosphere solution, but with 25 μL of s-NHS and EDC solution.

### Bacterial Culture Preparation

Pseudomonas aeruginosa (strain PA14 wild type) and Escherichia coli (strain MC4100, K-12, F-araD139Δ(argF-lac)U169 rspL150 relA1 fIbB5301 fruA25 deoC1 ptsF25) cultures were obtained by first streaking from a frozen culture stock onto LB Lennox agar (IBI Scientific) plates and incubated at 37° C. for 24 h. Individual colonies from these plates were used to inoculate 100 mL of LB in triplicate, and subsequently grown for 18 h at 37° C. and shaking at 230 rpm. The 18 h cultures were centrifuged at 5000 rpm for 5 minutes, then resuspended in fresh LB to reach an optical density at 600 nm (OD600) of 0.50 as measured by a BioChrom Colourwave CO7500 Colorimeter.

Dose-Response Curves: Carbenicillin disodium salt, gentamicin sulfate, and rifampicin stock solutions were prepared to a final concentration of 10 mg/mL in water for the former two and 20% (v/v) DMSO/H₂O for the latter. These stock solutions were added into 180 μL of E. coli or P. aeruginosa resuspension in a 96-well plate such that 9 separate 10-fold dilutions of each antibiotic starting at 1000 μg/mL were achieved. Vehicle controls using pure water for carbenicillin and gentamicin and 20% (v/v) DMSO/H₂O for rifampicin were also created in the same 96-well plate. These plates were then incubated at 37° C. for 24 h with 230 rpm shaking, after which OD₆₀₀ measurements were taken with a SpectraMax M2 Plate Reader.

Antibiotic Exposure: 40 ml cell resuspensions in 50 ml conical tubes were treated with specified concentration of antibiotics for the indicated time in a shaking incubator at 37° C. and 230 rpm.

Lysate Extraction: After the specified time of growth, these aliquots were then washed twice with phosphate buffer solution (pH=7.4) by centrifugation at 5000 rpm for 5 min and re-suspension in 40 mL PBS. The cell pellet was then resuspended in 100 μl of sterile millipore water and heated at 100° C. for 30 min. The resulting suspension was centrifuged at 12,000 rpm for 10 min, and the supernatant was collected and stored at −20° C. for subsequent SERS analysis.

### Metabolite Mixture Preparation

Metabolite mixtures are prepared as follows: 2-methyl napthalene (A), o-cresol (B), 2-amino acetophenone (C), Pyrrole (D), 2-pentyl furan (E), and Indole (F) are dissolved in ethanol at a concentration of 100 ppm. Then 1 ppm solutions are prepared in water from these ethanol stock solutions. The 63 combinations of metabolites are prepared by mixing the water stock solutions to maintain a total metabolite concentration of 1 ppm.

### SERS Spectroscopy

SERS spectroscopy measurements can be conducted using a confocal Renishaw InVia micro Raman system with a 785 nm diode laser, a laser power of 14 μW, an exposure time of 0.5 s, and a 60× water immersion objective with a 1.2 numerical aperture. Bacteria cell lysate or metabolite mixture solutions are used as the immersion media. After soaking the SERS substrate in the sample for 15 minutes, Raman maps are collected with a spacing of 4 μm spacing between points. For each sample one 20×20 pixel Raman map is acquired.

### SERS Spectra Processing

In some embodiments, SERS spectra can be pre-processed prior to training the VAE in three steps: 1) smoothing, 2) background subtraction, and 3) scaling. All three steps can be done using the Python 3.3 programming language. Smoothing can be done with the Savitzky-Golay method as implemented in Scikit-Learn using an 11 pixel window and polynomial order 3. Background subtraction can be done with the asymmetric least squares method and implemented in NumPy with λ=10000, p=0.001. Spectra can be scaled to have a minimum value of 0 and maximum value of 1 with Scikit-learn's minmaxscaler.

### Variational Autoencoder Implementation

Many embodiments include the training parameters of VAE models. All artificial neural network models are implemented in keras and use the adam optimizer. Prior to use in the variational autoencoder (VAE), SERS spectra are preprocessed. These 1011 dimensional spectra are padded with zeros to 1024 dimensions and reshaped to a dimension of (examples, 1024, 1) for use in 1 dimensional convolutional neural network (1D CNN) layers. All 1D CNN layers have a kernel window of 8 pixels, a stride of 2, are regularized with a maximum kernel norm of 3, have parametric relu activations, are batch normalized, and followed with a 30% dropout layer. Early stopping is implemented with test loss, and the batch size used is 32. VAE models use a loss function defined as KL divergence+mean absolute error*80. 400 spectra from each condition are used to train the VAE, with 20% of the spectra randomly removed from the training dataset and used as the test dataset. We do not condition the VAE space on condition labels (e.g. 0.5 hr control, 2 hr rifampicin, etc.), so we implement the VAE here as a fully unsupervised method.

The VAE is implemented differently for the antimicrobial susceptibility testing (AST) dataset and the AST and metabolite mixture combined dataset. For the smaller AST dataset, the encoder network is composed of 4 1D CNN layers with 32, 32, 64, and 64 filters. This output is flattened and sent to a 128 node fully connected layer with parametric relu activation, batch normalization, and 30% dropout and sent to a 32 node fully connected layer with parametric relu activation, and finally to fully connected layers with 2 nodes that represent the mean and standard deviation of the encoded input. The decoder is similar with a 1344 node fully connected layer, reshaped and sent to 4 1D transposed CNN layers with 64, 64, 32, and 32 filters. This is output to a 1D transposed CNN with stride 1, sigmoid activation, and a stride of 1.

For the larger combined dataset, the encoder network is composed of 6 1D CNN layers with 32, 32, 64, 64, 128, and 128 filters, with 40% dropout. This output is flattened and sent to a 256 node fully connected layer with parametric relu activation, batch normalization, and 50% dropout and sent to a 64 node fully connected layer with parametric relu activation, and finally to fully connected layers with 2 nodes that represent the mean and standard deviation of the encoded input. The decoder is similar with a 2048 node fully connected layer, reshaped and sent to 8 1D transposed CNN layers with 256, 256 (stride 1), 256, 256 (stride 1), 128, 128, 64, and 64 filters. This is output to a 1D CNN with 1 filter, sigmoid activation, and a stride of 1

### Semi-Supervised Learning

In some embodiments, The models are evaluated as follows. Examples are pulled from the test dataset used in training the VAE described above. These are used to train support vector machine (SVM) models with Scikit-learn using default settings. The SVM models are trained using preprocessed spectra with dimension 1011 and the accuracy is evaluated using the rest of the test dataset. The VAE SVM models were evaluated with the same examples projected into the latent space of the trained AST VAE and evaluated with the same dataset. This process is done 50 times and the mean and standard deviation of the model accuracy on the remaining spectra are depicted.

### Transfer Learning

Many embodiments involve the methods used in data informed transfer learning. The dataset used is the AST dataset encoded into a 2 dimensional latent space using the combined VAE. Outliers are removed by training an isolation forest on the training dataset and applying it to the training and test dataset. Isolation forest is implemented in Scikit-learn with the default settings and an outlier fraction of 5%. The outlier removed training dataset is then used to train a Bayesian Gaussian Mixture Model, which is implemented in Scikit-learn with the default settings and 6 components, and evaluated on the test dataset, which is plotted in FIG. 6a.

The neural network models are evaluated with categorical cross entropy loss and have the following architectures. First two models are trained for the transfer learning with the six unmixed metabolite full datasets. The first network is a deep CNN trained on the full dimensional preprocessed data and is composed of 4 1D CNN layers with parameters as above and filters of 16, 16, 32, and 32 that are followed by 50% dropout layers and batch normalized. This output is flattened and set to a 6 node fully connected layer with softmax activation. The second network is a multilayer perceptron trained on the VAE encoded, outlier removed spectra and is composed of 2 fully connected layers with 8 and 16 nodes and are batch normalized and with relu activation. This output is sent to a fully connected layer with 6 nodes and softmax activation. The weights of these trained networks are then fine-tuned with the few-example AST dataset. Additionally, these same models are evaluated with the same AST examples with standard Xavier initialization of the weights. The accuracy of these models are evaluated, and repeated 10 times to obtain a mean and standard deviation of the model accuracy and plotted.

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

What is claimed is:

1. A method of rapid antimicrobial susceptibility testing comprising:
obtaining a set of metabolic profile for at least one bacteria strain using a sensing platform;
generating a set of surface enhanced Raman scattering (SERS) spectra based upon the set of bacterial metabolic profile using at least one SERS sensor from the sensing platform;
evaluating at least one spectrum based on the set of SERS spectra using a machine learning model implemented on the sensing platform; and
when the at least one evaluated spectrum satisfies at least one criterion by the sensing platform, determining at least one antimicrobial susceptibility property of the at least one bacteria strain.

2. The method of claim 1, wherein the at least one bacteria strain is selected from the group consisting of Pseudomonas aeruginosa (P. aeruginosa), Escherichia coli (E. coli), uropathogenic strain of E. coli, Enterococcus faecalis (E. faecalis), Klebsiella pneumoniae (K. pneumoniae), co-culture of E. coli, K. pneumoniae, and P. aeruginosa, co-culture of E. coli and Salmonella enterica serovar Typhimurium (S. typhimurium), pairwise co-culture of uropathogenic strain of E. coli, E. faecalis, and K. pneumoniae.

3. The method of claim 1, wherein the at least one antimicrobial susceptibility property is selected from the group consisting of antibiotic susceptible metabolite profile, antibiotic resistance metabolite profile, antibiotic temporal response, and antibiotic dosage response.

4. The method of claim 3, wherein the determination of antibiotic dosage response is at least 10 times lower than a minimum inhibitory concentration.

5. The method of claim 1, wherein the machine learning model is selected from the group consisting of variational autoencoder (VAE), support vector machine (SVM), convolutional neural networks (CNNs), and Bayesian Gaussian mixture.

6. The method of claim 1, further comprising processing the set of SERS spectra by smoothing, background subtraction, and scaling.

7. The method of claim 1, wherein determining when the determined at least one antimicrobial susceptibility property satisfies at least one criterion further comprises:
generating a set of SERS spectra based upon the set of metabolic profile for each of the candidate bacteria strain;
determining at least one antimicrobial susceptibility property for each of the candidate bacteria strain based on the set of SERS spectra of each of the candidate bacteria strain using the machine learning model;
screening the candidate bacteria strain based upon the at least one antimicrobial susceptibility property determined for each of the candidate bacteria strain; and
identifying the antimicrobial susceptibility property based upon the screening.

8. The method of claim 1, further comprising training the machine learning model to learn relationships between the set of SERS spectra and antimicrobial susceptibility properties using a training dataset describing a plurality of bacteria strains and their antimicrobial susceptibility properties.

9. The method of claim 8, wherein training the machine learning model to learn relationships between the set of SERS spectra and antimicrobial susceptibility properties further comprises:
obtaining a set of SERS spectra for each bacteria strain in the training dataset of bacteria strains by determining a set of metabolic profile.

10. The method of claim 8, wherein training of the machine learning model is unsupervised, semi-supervised, supervised, or combinations thereof.

11. The method of claim 8, wherein the machine learning model is a variational autoencoder model and the set of SERS spectra is encoded in the VAE model in to a latent space as Gaussian distributions with mean and variance during training.

12. A method of training a machine learning model to predict at least one antimicrobial susceptibility property from a set of metabolic profile for a bacteria strain comprising:
obtaining a training dataset of bacteria strains and their antimicrobial susceptibility properties using a computer system;
generating a set of surface enhanced Raman scattering (SERS) spectra for each bacteai strain in the training dataset based upon a set of metabolic profile for each of the candidate bacteria strains using the computer system;
training a ML model to learn relationships between the set of SERS spectra of each bacteria strain in the training dataset and the antimicrobial susceptibility properties of each of the bacteria strains in the training dataset using the computer system; and
utilizing the machine learning model to predict at least one antimicrobial susceptibility property for a specific bacteria strain based upon a set of SERS spectra generated for the specific bacteria strain based upon a set of metabolic profile for the specific bacteria strain.

13. The method of claim 12, wherein training the machine learning model to learn relationships between the sets of SERS spectra of each bacteria strain in the training dataset and the antimicrobial susceptibility properties of each of the bacteria strains in the training dataset further comprises utilizing a transfer learning process to train a machine learning model previously trained to determine the relationship between a SERS spectrum of a bacteria strain and a different set of antimicrobial susceptibility properties.
